(19) United States
(12) Patent Application Publication
(10) Pub. No.: US 2020/0179417 A1
(43) Pub. Date: Jun. 11, 2020

(54) COMBINATION CHEMOTHERAPIES

(71) Applicants: Spyro MOUSSES, Scottsdale, AZ (US); Systems Oncology, LLC, Scotsdale, AZ (US)

(72) Inventor: Spyro Mousses, Scottsdale, AZ (US)

(73) Assignee: Systems Oncology, LLC, Scotsdale, AZ (US)

(21) Appl. No.: 16/313,013

(22) PCT Filed: Jun. 23, 2017
(86) PCT No.: PCT/US2017/038964

§ 371 Date: Dec. 22, 2018

Related U.S. Application Data
(60) Provisional application No. 62/355,293, filed on Jun. 27, 2016.

(51) Int. Cl. A61K 31/69; A61K 31/122; A61K 31/655; A61K 31/352; A61K 45/06; A61K 31/4375; A61K 31/131

(52) U.S. Cl. CPC A61K 31/69; A61K 31/122; A61K 31/655; A61K 31/131; A61K 45/06; A61K 31/4375; A61K 31/352

(57) ABSTRACT

Combination of agents that increase the amount of reactive oxygen species with agents that are activated, enhanced, or induced by oxygen species for the treatment of cancer and pre-cancerous disease. Pharmaceutical compositions comprising a therapeutic agent or drug that generate or produce reactive oxygen species (ROS) in a disease microenvironment, and at least one drug or agent that is activated, enhanced, or induced by ROS for the treatment of mammalian cancer, dysplastic disorders, neoplastic, or hyperproliferative disorders and methods of using thereof for the treatment of mammalian cancer dysplastic disorders, neoplastic, or hyperproliferative disorders.

### FIELD OF THE INVENTION

This invention relates to pharmaceutical compositions, methods, and approaches comprising a therapeutic agent that creates ROS in a disease microenvironment and at least one chemotherapeutic drug or pro-drug that is activated, enhanced, or induced by ROS for the treatment of mammalian cancer or hyperproliferative disorders, including a wide range of hyperplastic, dysplastic, and neoplastic disorders. This invention also relates to methods of treating mammalian cancer or hyperproliferative disorders, said method comprising contacting cancer cells or any other hyperproliferative cells with said compositions.

### BACKGROUND OF THE INVENTION

Cancer is the most common cause of death in many parts of the world and over 2.5 million cases of cancer are diagnosed globally every year. In spite of the exponentially expanding number of cancer therapeutics available, the high toxicity and increasingly high specificity of such drugs means there is a continuing need for new anti-cancer agents and strategies, particularly those that have fewer toxic side-effects and are efficacious against a broad spectrum of cancers. Thus new fundamental treatment paradigms that are more cancer selective are required. The present invention provides such a breakthrough strategy, compositions and methods.

It is widely recognized that employing a single treatment strategy against cancer is generally ineffective due to the multi-factorial nature of this disease. The combination of more than one drug to maximize the anticancer response is being increasingly utilized. See Gene Ther., 2000, vol. 11, 1852.

More than 30 different drugs are commonly used for chemotherapy. The most effective of these drugs, known as first-line drugs, are doxorubicin, epirubicin, methotrexate, cyclophosphamide, 5-fluorouracil, docetaxel and paclitaxel. Although each of these individual drugs has shown some efficacy on its own, Applicants' research has shown that combining different drugs further increases their ability to kill cancer cells. Some of the currently available combinations of chemotherapy are:
1. a combination of cyclophosphamide and doxorubicin (Adriamycin).
2. a combination of cyclophosphamide, methotrexate and 5-fluorouracil.
3. CAF (FAC), a combination of cyclophosphamide, doxorubicin (Adriamycin) 5-fluorouracil.
4. a combination of cyclophosphamide, doxorubicin (Adriamycin) and paclitaxel (Taxol).
5. a combination of cyclophosphamide, doxorubicin (Adriamycin) and taxotere (Docetaxel).
   Overall, there are many cases where known chemotherapeutic agents fail to eradicate cancer due to acquired resistance of the cancer to the agent. According to the instant invention, compounds in combination with another chemotherapeutic agent may be administered at a dose lower than the current standard while still providing beneficial efficacy and perhaps reducing toxicity of the chemotherapeutic agent to the patient.

### SUMMARY OF THE INVENTION

This invention relates to a pharmaceutical compositions comprising at least one first compound that increases the amount of reactive oxygen species in a disease microenvironment and at least one second compound that is activated, enhanced, or induced by reactive oxygen species. The invention includes such compositions, plus pharmaceutically acceptable carriers or diluents; methods of killing tumor cells in humans afflicted therewith which comprises administering to such humans an effective tumor cell killing amount of such pharmaceutical compositions.

### Definitions

The definitions and terminology used herein is for the purpose of describing particular embodiments only and is not intended to be limiting.

Definitions of specific functional groups and chemical terms are described in more detail below. General principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

As used in the specification and the appended claims, the singular forms “a,” “and” and “the” include plural references unless the context clearly dictates otherwise.

For the recitation of numeric ranges herein, each intervening number with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “about” is used synonymously with the term “approximately.” Illustratively, the use of the term “about” indicates that values slightly outside the cited values, namely, plus or minus 10%. Such values are thus encompassed by the scope of the claims reciting the terms “about” and “approximately.”

The term “comprising” and “including” are used in their open, non-limiting sense.

The terms “abnormal cell growth” and “hyperproliferative disorder” are used interchangeably in this application.

“Abnormal cell growth” refers to cell growth that is independent of normal regulatory mechanisms (e.g., loss of contact inhibition), including the abnormal growth of normal cells and the growth of abnormal cells.

As used herein, the term “activated” means rendered more reactive or useful according to the invention.

The term “acyl” includes alkyl, aryl, or heteroaryl substituents attached to a compound via a carbonyl functionality (e.g., —C(O)-alkyl, —C(O)-aryl, etc.). Examples are an alkylcarbonyl, cycloalkylcarbonyl, heterocyclylcarbonyl, arylcarbonyl or heteroarylcarbonyl substituent, any of which may be further substituted (e.g., with one or more substituents).

The term “acylamino” refers to an acyl radical appended to an amino or alkylamino group, and includes —C(O)—NH2 and —C(O)—NRR′ groups where R and R′ are as defined in conjunction with alkylamino.

The term “acyloxy” refers to the ester group —OC(O)—R, where R is H, alkyl, alkenyl, alkynyl, or aryl.

“Administration” or “administering,” as used herein, refers to providing, contacting, and/or delivery of a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

The term “alkenyl” includes alkyl moieties having at least one carbon-carbon double bond, including E and Z isomers of said alkenyl moiety. The term also includes cycloalkyl moieties having at least one carbon-carbon double bond, i.e., cycloalkenyl. Examples of alkenyl radicals include ethenyl, propenyl, butenyl, 1,4-butadienyl, cyclopentenyl, cyclohexenyl, prop-2-enyl, but-2-enyl, but-3-enyl, 2-methylprop-2-enyl, hex-2-enyl, and the like. An alkenyl group may be optionally substituted. Examples of alkenyl groups include, but are not limited to, allyl, propenyl, 2-butenyl, 3-hexenyl and 3-octenyl groups. One of the double bond carbons may optionally be the point of attachment of the alkenyl substituent.

The term “alkenylene” refers to a divalent alkenyl, e.g., —CH—CH—, —CH—CH₂CH₂— or —CH—C—CH—. An alkenylene may be optionally substituted.

The term “alkenylene” refers to a divalent straight chain, branched chain or cyclic saturated aliphatic group containing at least one carbon-carbon double bond, and including E and Z isomers of said alkenylene moiety. An alkyenylene group may be optionally substituted.

The term “alkoxy” means an O-alkyl group. Examples of alkoxy radicals include methoxy, ethoxy, n-propoxy, isopropoxy, n-butoxy, iso-butoxy, sec-butoxy, tert-butoxy and the like.

The term “alkyl” refers to a straight or branched hydrocarbon chain, containing the indicated number of carbon atoms. It further means, saturated monovalent hydrocarbon radicals having straight, cyclic or branched moieties. An “alkyl” group may include an optional carbon-carbon double or triple bond where the alkyl group comprises at least two carbon atoms. Cycloalkyl moieties require at least three carbon atoms. Examples of straight or branched alkyl radicals include methyl (Me), ethyl (Et), n-propyl, isopropyl, n-butyl, isobutyl, sec-butyl, tert-butyl, tert-amyl, pentyl, isopentyl, hexyl, heptyl, octyl and the like. An alkyl group may be optionally substituted.

The term “alkylamino” refers to the —NRR′ group, where R and R′ are independently selected from hydrogen (however, R and R′ cannot both be hydrogen), alkyl, and aryl groups; or R and R′, taken together, can form a cyclic ring system.

The term “alkylene” refers to a divalent straight chain, branched chain or cyclic saturated aliphatic group. The latter group may also be referred to more specifically as a cycloalkylene group. Further, “alkylene” refers to a divalent alkyl, e.g., —CH₂—, —CH₂CH₂—, —CH₂CH₂CH₂— or —CH₂CH(CH₃)CH₂—. An alkylene group may be optionally substituted.

The term “alkylthio” alone or in combination, refers to an optionally substituted alkyl thio radical, alkyl-S—.

The term “alkynyl” refers to straight- and branched-chain alkynyl groups having from two to twelve carbon atoms, preferably from 2 to 6 carbons, and more preferably from 2 to 4 carbons. Examples of alkynyl groups include, but are not limited to, ethynyl, propargyl, and 3-hexynyl. One of the triple bond carbons may optionally be the point of attachment of the alkynyl substituent.

The term “alkynylene” refers to a divalent alkynyl, e.g., —C≡C— or —C≡C—CH₂—. An alkynyl or alkynylene may be optionally substituted.

The term “amide” refers to the radical —C(O)N(R′)(R″) where R′ and R″ are each independently selected from hydrogen, alkyl, alkenyl, alkynyl, —OH, alkoxy, cycloalkyl, heterocycloalkyl, heteroaryl, aryl as defined above; or R′ and R″ cyclize together with the nitrogen to form a heterocycloalkyl or heteroaryl.

The term “amino” refers to a group of the formula —NR¹R², wherein R¹ and R² are each independently selected from, for example, hydrogen, alkyl, cycloalkyl, heterocyclyl, aryl and heteroaryl, or R¹ and R², together with the nitrogen to which they are attached, may form a ring structure. Examples of amino groups include, but are not limited to, —NH₂, alkylamino groups such as —NHCH₃, —NHCH₂CH₃ and —NHCH(CH₃)₂, dialkylamino groups such as —N(CH₃)₂ and —N(CH₂CH₃)₂, and arylamino groups such as —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The groups R¹ and R² may be optionally substituted.

“Amino acid” refers to any of the naturally occurring amino acids, as well as synthetic analogs and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). See, e.g., Harper et al (1977) Review of Physiological Chemistry, 16th Ed., Lange Medical Publications, pp. 21-24. One of skill in the art will appreciate that the term “amino acid” also includes β-, γ-, δ-, and ω-amino acids, and the like. Unnatural amino acids are also known in the art, as set forth in, for example, Williams (ed.), Synthesis of Optically Active α-Amino Acids, Pergamon Press (1989); Evans et al., J. Amer. Chem. Soc., 112:4011-4030 (1990); Pu et al., J. Amer. Chem. Soc., 56:1280-1283 (1991); Williams et al., J. Amer. Chem. Soc., 113:9276-9286 (1991); and all references cited therein.

As used herein, the twenty conventional amino acids and their abbreviations follow conventional usage (see IMMUNOLOGY-A SYNTHESIS, 2nd Edition, E. S. Golub and D. R. Gren, Eds., Sinauer Associates, Sunderland, Mass. (1991), which is incorporated herein by reference). Amino acid residues are abbreviated as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Stereoisomers (e.g., D-amino acids) of the twenty conventional amino acids, unnatural amino acids such as α,α-disubstituted amino acids, N-alkyl amino acids, and other unconventional amino acids may also be suitable components for compounds of the present invention. Examples of unconventional amino acids include: 4-hydroxyproline, O-phosphoserine, N-acetylserine, N-formylmethionine, 3-methylhistidine, 5-hydroxylysine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

The term “anti-neoplastic agent” refers to agents capable of inhibiting or preventing the growth of neoplasms, or checking the maturation and proliferation of malignant (cancer) cells.

The term “aromatic” refers to compounds or moieties comprising multiple conjugated double bonds. Examples of aromatic moieties include, without limitation, aryl or heteroaryl ring systems.

“Aryl” or “Ar” refers to an unsaturated aromatic carbocyclic group of from 6 to 14 carbon atoms having a single ring (e.g., phenyl) or multiple condensed rings (e.g., naphthyl or anthryl) carbon atoms, which can optionally be unsubstituted or substituted with from 1 to 3 substituents selected from hydroxy, alkyl, substituted alkyl, alkoxy, substituted alkoxy, amido, amino, aryloxy, carboxyl, halo, mercapto, cyano, nitro, —SO₃, —SO₂NH₂ and other non-interfering substituents. Preferred aryls include phenyl and alkyl substituted phenyl. Preferred aryl groups have from 4 to 20 ring atoms, and more preferably from 6 to 14 ring atoms. An aryl group may be optionally substituted. Examples of aryl moieties include, but are not limited to, phenyl, naphthyl, and anthracenyl.

The term “arylalkyl” refers to an alkyl moiety in which an alkyl hydrogen atom is replaced with an aryl group. Arylalkyl includes groups in which more than one hydrogen atom has been replaced with an aryl group. Examples of arylalkyl groups include benzyl, 2-phenylethyl, 3-phenylpropyl, 9-fluorenyl, benzhydryl, and trityl groups.

The term “aryloxy” means aryl-O—.

The term “arylthio” means an aryl thio radical, aryl-S—.

The term “carbamoyl” or “carbamate” refers to the group —O—C(O)—NRR″ where R and R″ are independently selected from hydrogen, alkyl, and aryl groups; and R and R″ taken together can form a cyclic ring system.

The term “carbocyclyl” includes optionally substituted cycloalkyl and aryl moieties. The term “carbocyclyl” also includes cycloalkenyl moieties having at least one carbon-carbon double bond.

The term “carboxy esters” refers to —C(O)OR where R is alkyl or aryl.

As used herein, the term “carcinomas” refers to lesions that are cancerous. Examples include malignant melanomas, breast cancer, prostate cancer and colon cancer.

“Contacting,” as used herein as in “contacting a cell,” refers to contacting a cell directly or indirectly in vitro, ex vivo, or in vivo (i.e. within a subject, such as a mammal, including humans, mice, rats, rabbits, cats, and dogs). Contacting a cell, which also includes “reacting” a cell, can occur as a result of administration to a subject. Contacting encompasses administration to a cell, tissue, mammal, subject, patient, or human. Further, contacting a cell includes adding an agent to a cell culture. Other suitable methods may include introducing or administering an agent to a cell, tissue, mammal, subject, or patient using appropriate procedures and routes of administration as defined herein.

The term “cycloalkyl” refers to a monocyclic or polycyclic radical which contains only carbon and hydrogen, and may be saturated, partially unsaturated, or fully unsaturated. A cycloalkyl group may be optionally substituted. Preferred cycloalkyl groups include groups having from three to twelve ring atoms, more preferably from 5 to 10 ring atoms. Any ring atom can be substituted (e.g., with one or more substituents). Cycloalkyl groups can contain fused rings. Fused rings are rings that share one or more common carbon atoms. Examples of cycloalkyl groups include, but are not limited to, cyclopropyl, cyclobutyl, cyclopentyl, cyclohexyl, cyclohexenyl, cyclohexadienyl, methylcyclohexyl, adamantyl, norbornyl and norbornenyl.

“Effective amount,” as used herein, refers to a dosage of the compounds or compositions effective for eliciting a desired effect. This term as used herein may also refer to an amount effective at bringing about a desired in vivo effect in an animal, mammal, or human, such as reducing proliferation of a cancer cell.

The terms “enhance” or “enhancing” or “enhanced” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system (e.g., a tumor cell). An “enhancing-effective amount,” as used herein, refers to an amount adequate to enhance the effect of another therapeutic agent in a desired system (including, by way of example only, a tumor cell in a patient). When used in a patient, amounts effective for this use depends on the severity and course of the proliferative disorder (including, but not limited to, cancer), previous therapy, the patient's health status and response to the drugs, and the judgment of the treating physician. It is considered well within the skill of the art for one to determine such enhancing-effective amounts by routine experimentation.

“Ester” or “carboxyl ester” refers to the group —C(O)OR where R is alkyl, aryl, arylalkyl, or heteroaryl (including substituted alkyl, substituted aryl, substituted heteroaryl, or substituted arylalkyl).

An “excipient” generally refers to substance, often an inert substance, added to a pharmacological composition or otherwise used as a vehicle to further facilitate administration of a compound. Examples of excipients include but are not limited to calcium carbonate, calcium phosphate, various sugars and types of starch, cellulose derivatives, gelatin, vegetable oils and polyethylene glycols.

The term “halo” or “halogen” means fluoro, chloro, bromo or iodo.

The terms haloalkyl, haloalkenyl, haloalkynyl and haloalkoxy include alkyl, alkenyl, alkynyl and alkoxy structures, that are substituted with one or more halo groups or with combinations thereof.

The terms “heteroalkyl” “heteroalkenyl” and “heteroalkynyl” include optionally substituted alkyl, alkenyl and alkynyl radicals and which have one or more skeletal chain atoms selected from an atom other that carbon, e.g., oxygen, nitrogen, sulfur, phosphorus or combinations thereof.

The term “heteroaryl” as used herein refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms independently selected from O, N, S, P and Si (e.g., carbon atoms and 1-3, 1-6, or 1-9 heteroatoms independently selected from O, N, S, P and Si if monocyclic, bicyclic, or tricyclic, respectively). Any ring atom can be substituted (e.g., with one or more substituents). Heteroaryl groups can contain fused rings, which are rings that share one or more common atoms. Examples of heteroaryl groups include, but are not limited to, radicals of pyridine, pyrimidine, pyrazine, pyridazine, pyrrole, imidazole, pyrazole, oxazole, isoxazole, furan, thiazole, isothiazole, thiophene, quinoline, isoquinoline, quinoxaline, quinazoline, cinnoline, indole, isoindole, indolizine, indazole, benzimidazole, phthalazine, pteridine, carbazole, carboline, phenanthridine, acridine, phenanthroline, phenazine, naphthyridines and purines.

The term “heterocyclyl” refers to aromatic and non-aromatic heterocyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl. An example of a 6 membered heterocyclic group is pyridyl, and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (—O) moieties such as pyrrolidin-2-one. A heterocyclyl group may be optionally substituted.

“Heterocycle” or “heterocyclic” refers to a monovalent saturated, unsaturated or aromatic (heteroaryl) carbocyclic group having a single ring or multiple condensed rings having at least one hetero atom, such as nitrogen, sulfur or oxygen within the ring, which can optionally be unsubstituted or substituted with from 1 to 3 substituents selected from the group consisting of hydroxy, alkyl, substituted alkyl, alkoxy, substituted alkoxy, aryl, substituted aryl, halo, mercapto, and other non-interfering substituents.

A “heterocycloalkyl” group refers to a cycloalkyl group that includes at least one heteroatom selected from nitrogen, oxygen and sulfur. The radicals may be fused with an aryl or heteroaryl.

The term “hydroxy” refers to an —OH radical. The term “alkoxy” refers to an —O— alkyl radical.

The term “induced” means stimulated or increased.

By the term “inhibiting the growth of tumor cells” as used herein is meant the inhibition of the growth of tumor cells which are sensitive to the method of the subject invention, i.e., therapy involving the administration of an effective amount of the combination of a compound of the camptothecin class, such as topotecan, and a platinum coordination compound, such as cisplatin to a human afflicted therewith. Preferably such treatment also leads to the regression of tumor growth, i.e., the decease in size of a measurable tumor. Most preferably, such treatment leads to the complete regression of the tumor.

The term “lower alkoxy” refers to alkoxy groups having from 1 to 8 carbons, including straight, branched or cyclic arrangements.

The term “lower alkylmercapto” refers to a sulfide group that is substituted with a lower alkyl group; and the term “lower alkyl sulfonyl” refers to a sulfone group that is substituted with a lower alkyl group.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “mercapto” or “thiol” refers to an —SH radical.

The term “neoplasm” is defined as in Stedman's Medical Dictionary 25th Edition (1990) and refers to an abnormal tissue that grows by cellular proliferation more rapidly than normal and continues to grow after the stimuli that initiated the new growth ceases. Neoplasms show partial or complete lack of structural organization and functional coordination compared with normal tissue, and usually form a distinct mass of tissue that may be either benign (benign tumor) or malignant (cancer).

“Optional” or “optionally” means that the subsequently described event or circumstance may or may not occur, and that the description includes instances where said event or circumstance occurs and instances in which it does not. “Optionally substituted” groups may be substituted or unsubstituted. When substituted, the substituents of an “optionally substituted” group may include, without limitation, one or more substituents independently selected from the following groups or designated subsets thereof: (C1-C6)alkyl, (C2-C6)alkenyl, (C2-C6)alkynyl, (C1-C6)heteroalkyl, (C1-C6)haloalkyl, (C2-C6)haloalkenyl, (C2-C6)haloalkynyl, (C3-C6)cycloalkyl, phenyl, (C1-C6)alkoxy, phenoxy, (C1-C6)haloalkoxy, amino, (C1-C6)alkylamino, (C1-C6)alkyIthio, phenyl-S—, oxo, (C1-C6)carboxyester, (C1-C6)carboxamido, (C1-C6)acyloxy, H, halogen, CN, NO2, NH2, N3, NHCH3, N(CH3)2, SH, SCH3, OH, OCH3, OCF3, CH3, CF3, C(O)CH3, CO2CH3, CO2H, C(O)NH2, pyridinyl, thiophene, furanyl, (C1-C6)carbamate, and (C1-C6)urea. An optionally substituted group may be unsubstituted (e.g., —CH2CH3), fully substituted (e.g., —CF2CF3), monosubstituted (e.g., —CH2CH2F) or substituted at a level anywhere in-between fully substituted and monosubstituted (e.g., —CH2CF3).

The term “oxo” means an “O” group.

As used herein the term “patient” refers to someone who is or has been under the care of a health care provider, including without limitation, doctors, nurses, naturopaths, therapists, chiropractors, and the like, with respect to the disease or condition at issue.

The term “perhalo” refers to groups wherein every C—H bond has been replaced with a C-halo bond on an aliphatic or aryl group. Examples of perhaloalkyl groups include —CF3 and —CFCl2.

The term “pharmaceutically acceptable” means pharmacologically acceptable to the subject being administered the agent. As would be understood by one skilled in the art, In the case of cytotoxic oncology agents, such term may include agents that may have considerable toxicity towards healthy subjects or cells as the case may be.

A “pharmacological composition” refers to a mixture of one or more of the compounds described herein, or physiologically acceptable salts thereof, with other chemical components, such as physiologically acceptable carriers and/or excipients. The purpose of a pharmacological composition is to facilitate administration of a compound to an organism.

A “physiologically acceptable carrier” refers to a carrier or diluent that does not cause or unacceptable irritation to an organism and does not unacceptably abrogate the biological activity and properties of the administered compound. As would be understood by one skilled in the art, In the case of cytotoxic oncology agents, such term may include agents that may have considerable toxicity towards healthy subjects or cells as the case may be.

As used herein and in the claims, “pharmaceutically acceptable salts” refer to derivatives of the disclosed compounds that are modified by making acid or base salts, or by modifying functional groups present in the compounds in such a way that the modifications are cleaved, either in routine manipulation or in vivo in relation to the parent compounds.

Examples include, but are not limited to, mineral or organic acid salts of basic residues such as amines; alkali or organic salts of acidic residues such as carboxylic acids; acetyl formyl and benzoyl derivatives of amines; and the like.

Pharmaceutically-acceptable salts of the compounds of the invention are prepared by reacting the free acid or base forms of these compounds with a stoichiotretric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference in its entirety.

The salts can be prepared in situ during the final isolation and purification of such compounds, or separately by reacting the free base or acid functions with a suitable organic acid or base, for example. Representative acid addition salts include the hydrochloride, hydrobromide, sulfate, bisulfate, acetate, valerate, oleate, palmatate, stearate, laurate, borate, benzoate, lactate, phosphate, tosylate, mesylate, citrate, maleate, fumarate, succinate, tartrate, glucoheptonate, lactobionate, lauryl sulfate salts and the like. Representative alkali and alkaline earth metal salts include the sodium, calcium, potassium and magnesium salts.

“A pharmaceutically acceptable salt” is intended to mean a salt that retains the biological effectiveness of the free acids and bases of the specified compound and that is not biologically or otherwise undesirable.

A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, di hydrogen phosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, phosphoric acid and the like, or with an organic acid, such as acetic acid, phenylacetic acid, propionic acid, stearic acid, lactic acid, ascorbic acid, maleic acid, hydroxymaleic acid, isethionic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid, 2-acetoxybenzoic acid or cinnamic acid, a sulfonic acid, such as p-toluenesulfonic acid, methanesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine and piperazine, and inorganic salts derived from sodium, calcium, potassium, magnesium, manganese, iron, copper, zinc, aluminum and lithium.

“Prodrugs” are inactive or lower activity derivatives or variants of bioactive molecules that are designed to become more activate in vivo by a variety of stimuli. The success of the prodrug approach is illustrated by the fact that about 10% of clinical therapeutics are classified as prodrugs. Prodrug strategies may serve to improve the drug-like properties of bioactive molecules including bioavailability, cell permeability, and pharmacokinetics.

As used herein the term “reactive oxygen species” or “ROS” are a class of oxygen-containing chemical compounds that may be defined by their reactivity towards biological targets, including lipids, proteins and DNA. The most prominent member of this class is the superoxide anion (0-2). The 0-2 is rapidly converted to H₂O₂ by distinct superoxide dismutases (SODs) or to hydroxyl radicals (OH.). While 0-2 released into the mitochondrial matrix is directly converted by SOD2 into the less reactive H2O2, O-2 released into the mitochondrial inter-membrane space can be exported via voltage-dependent anion channels (VDAC) into the cytosol followed by a SOD1-mediated conversion into H2O2. In addition, cellular membrane-associated NOXs transferring electrons from NAD(P)H across cell membranes to molecular oxygen (O2) are producers of superoxide anions. The hydroxyl radical (OH.) is considered the most reactive ROS species. Due to its high reactivity towards lipids, proteins and DNA, it has a short half-life thereby limiting its diffusion but causing damage largely at its site of production. Lennicke et al. Cell Communication and Signaling (2015) 13:39.

As used herein, the term “subject” is intended to include human and non-human animals. Exemplary human subjects include a human patient having a disorder, e.g., cancer, or a normal subject. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

The term “substituted” means that the group in question, e.g., alkyl group, etc., may bear one or more substituents. For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— optionally also recites —OCH₂—.

In accordance with a convention used in the art, the group:

[symbol]

is used in structural formulas herein to depict the bond that is the point of attachment of the moiety or substituent to the core or backbone structure.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such substituent. Combinations of substituents and/or variables are permissible, but only if such combinations result in stable compounds.

For example, the substituted aryl and heteroaryl groups of the invention have one or more hydrogen atoms replaced with a halo, hydroxy, alkyl, alkoxy, amino, cyano, carboxy, carbalkoxy, nitro or trifluoromethyl group. A halo group is a halogen, and includes fluoro, chloro, bromo and iodo groups. The term alkoxy refers to an alkyl group having at least one oxygen substitutent. The term carbalkoxy refers to groups of the formula —R—C(O)O— where R is an alkyl group.

“Substituted alkoxy” refers to —O-substituted alkyl and includes, by way of example, —OCF₃, —OCH₂-ϕ and the like.

The term “—SO₂ (lower alkyl)” refers to a sulfonyl group that is substituted with a lower alkyl group.

The compositions containing the compound(s) of the described herein can be administered for prophylactic and/or therapeutic treatments. In therapeutic applications, the compositions are administered to a patient already suffering from a proliferative disorder or condition (including, but not limited to, cancer), as described above, in an amount sufficient to treat or at impact the symptoms of the proliferative disorder or condition. If not specifically defined herein, an amount adequate to accomplish this is defined as “therapeutically effective amount or dose.” Amounts effective for this use depends on the severity and course of the proliferative disorder or condition, previous therapy, the patient's health status and response to the drugs, and the judgment of the treating physician. In prophylactic applications, compositions containing the compounds described herein are administered to a patient susceptible to or otherwise at risk of a particular proliferative disorder or condition.

Except as specifically defined herein, such an amount is defined to be a “prophylactically effective amount or dose.” In this use, the precise amounts also depend on the patient's state of health, weight, and the like. It is considered well within the skill of the art for one to determine such therapeutically effective or prophylactically effective amounts by routine experimentation (e.g., a dose escalation clinical trial. The amount of a given agent that corresponds to such an amount varies depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated.

The term “thioalkoxy” or “thioether” refers to an —S-alkyl radical. The term “thioaryloxy” refers to an —S-aryl radical.

As used herein, the term “treat” or “treating” a subject having a disorder refers to administering a regimen to the subject, e.g., the administration of a compound or a composition comprising a compound, such that at least one symptom of the disorder is cured, healed, alleviated, relieved, altered, impacted, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “ureyl” or “urea” refers to the group —N(R)—C(O)—NR′ R″ where R, R′, and R″ are independently selected from hydrogen, alkyl, aryl; and where each of R—R′, R′—R″, or R—R″ taken together can form a cyclic ring system.

### DETAILED DESCRIPTION OF THE INVENTION

The present invention involves new anti-cancer agents and strategies, particularly those that have fewer toxic side-effects and are efficacious against a broad spectrum of cancers. Thus new fundamental treatment paradigms are required. The present invention provides such a break through strategy, compositions and methods.

The methods described herein can be used with any cancer, for example a carcinoma, a sarcoma, a myeloma, a leukemia, a lymphoma or a mixed type. Exemplary cancers include leukemia, non-small cell lung cancer, colon cancer, CNS cancer, melanoma, ovarian cancer, renal cancer, prostate cancer and breast cancer.

Tumor selectivity is necessary but often not sufficient for ameliorating cancer growth. There is an unmet need for combinations that synergize at the mechanistic level to selectively increase local toxicity locally in the cancer microenvironment, while not increasing general toxicity in non-disease tissues.

Low ROS levels is relevant for the maintenance of cellular homeostasis in normal cells, but most cancer cells show metabolic alterations resulting in significantly higher ROS levels, which can trigger pro- as well as anti-tumorigenic processes. The increased levels of ROS can promote pro-survival and pro-proliferative pathways as well as the metabolic adaption of tumor cells to the tumor environment. Lennicke et al. Cell Communication and Signaling (2015) 13:39. The instant invention manipulates these natural phenomena in order to promote the anti-tumorigenic potential of ROS.

Hydrogen peroxide (H₂O₂) is a key member of the class of reactive oxygen species (ROS), which are byproducts of the cellular metabolism including protein folding. Unlike the superoxide anion and hydroxyl radical, the less reactive H₂O₂ is involved in many physiological processes such as hypoxic signal transduction, cell differentiation and proliferation but also plays a role in mediating immune responses. H₂O₂ exerts its effects depending on the cellular context, its local concentration as well as its exposure time. Thus H₂O₂ is often considered an unwanted rather toxic byproduct, but plays an important role in the control of vital cellular processes. Lennicke et al. Cell Communication and Signaling (2015) 13:39. The manipulation of the increased ROS, especially hydrogen peroxide, in tumor cells provides the basis for the instant invention.

Several oncogenes, such as RAS, MYC and AKT as well as mutations or loss of tumor suppressors like p53, are associated with increased ROS levels. In addition, increased spatially localized ROS levels promotes cell toxicity thereby leading to the activation of cell cycle arrest or cell death-inducing pathways resulting in the inhibition of cancer progression. Lennicke et al. Cell Communication and Signaling (2015) 13:39.

There is a class of anti-cancer agents that use cancer cell enzymes to generate large amounts of ROS in and around cancer cells as a mechanism of inducing cancer cell death. This type of agent (referred to herein as “Producers of ROS”) includes the recent development of β-lapachone, which exploits the elevated levels of NAD(P)H:quinone oxidoreductase 1 (“NQO1”) in most solid tumors. Although in itself, this represents a novel and cancer selective chemotherapeutic approach, the level of cell death can be limited. Surprisingly, the combination of these types of agents with additional compounds that exploit the local effects on the microenvironment synergistically enhances cancer cell death beyond single agent treatments.

β-lapachone and similar analogues can be considered quinone prodrugs according to the present invention. Analogues of β-lapachone useful according to the present invention include those disclosed by U.S. Pat. Nos. 8,614,228; 8,067,459; 7,812,051; 7,790,765; 7,361,691; 7,074,824; 6,245,807; 5,969,163; 5,824,700; 5,763,625; and U.S. patent application Ser. No. 13/825,524 all disclose β-lapachone related molecules useful according to the present invention and are hereby incorporated by reference in their entirety.

Compositions according to the present inventions comprise compounds that are substrates for detoxifying enzymes such as NAD(P)H quinone oxidoreductase (NQO1, DT diaphorase). NQO1 is an FAD-dependent 2-electron reductase whose primary function is to protect the cell from cytotoxins, especially quinones. It is a Phase II detoxifying enzyme, the expression of which is regulated by NRF-2 and the antioxidant response element (ARE) in response to electrophilic or oxidative stress. Thus compounds in the quinone class of compounds or quinone-containing molecules comprise one aspect of the present invention.

Other Producers of ROS include agents like Deoxynyboquinone, which is a potent antineoplastic agent known as an anthraquinone, synthesized in seven linear steps through a route employing three palladium-mediated coupling reactions. Experiments performed on cancer cells grown in hypoxia and normoxia strongly suggest that DNQ undergoes bioreduction to its semiquinone, which then is re-oxidized by molecular oxygen, forming superoxide that induces cell death.

Furthermore, global transcript profiling of cells treated with DNQ shows elevation of transcripts related to oxidative stress, a result confirmed at the protein level by Western blotting. In contrast to most other antineoplastic agents that generate reactive oxygen species, DNQ potently induces death of cancer cells in culture, with IC(50) values between 16 and 210 nM. In addition, unlike the experimental therapeutic elesclomol, DNQ is still able to induce cancer cell death under hypoxic conditions. Thus DNQ therapeutics provide direct ROS generation as an anticancer strategy. J Am Chem Soc. 2010 Apr. 21; 132(15):5469-78. doi: 10.1021/ja100610m. Chemistry and biology of deoxynyboquinone, a potent inducer of cancer cell Death. Bair J S, Palchaudhuri R, Hergenrother P J.

Deoxynyboquinone kills a wide spectrum of cancer cell types (i.e., breast, non-small-cell lung, prostate, pancreatic) in an NQO1-dependent manner with greatly improved (20- to 100-fold) potency compared to β-lapachone. DNQ lethality relies on NQO1-dependent futile redox cycling, using oxygen and generating extensive reactive oxygen species, particularly superoxide and hydrogen peroxide. Elevated ROS levels cause extensive DNA lesions and PARP-1 hyperactivation that, in turn, results in severe NAD⁺/ATP depletion that stimulates calcium-dependent programmed necrotic cell death responses unique to this class of NQO1 ‘bioactivated’ drugs (i.e., β-lapachone and DNQ). J Am Chem Soc. 2010 Apr. 21; 132(15):5469-78. doi: 10.1021/ja100610m. Chemistry and biology of deoxynyboquinone, a potent inducer of cancer cell Death. Bair J S, Palchaudhuri R, Hergenrother P J.

According to a preferred embodiment of the present invention, compounds of the quinone class, such as DNQ which leads to an increased local production of ROS in the microenvironment of cancer, when administered in combination with compounds that are enhanced, activated, or induced by ROS in a tumors' microenviroment, exhibits surprisingly effective, synergistic, tumor cell destroying effects of the NQO1 substrate. It has also now been found that such synergism results in a need for lower doses of such compounds when administered with a compound of the quinone class as compared to the doses required when either such compound is administered without a compound of the other class.

In addition, U.S. Pat. No. 9,233,960 titled “Compounds and Anti-Tumor NQO1 Substrates” incorporated in its entirety herein by reference, discloses numerous examples of analogues of DNQ that are useful in the present invention.

As previously stated, reducing host toxicity is one of the main challenges of cancer chemotherapy. Many tumor cells contain high levels of ROS resulting from high levels of the detoxifying enzymes like NQO1. The over expression of these enzymes and resulting over abundance of ROS in tumor cells make them distinctively different from normal cells which do not have high levels of ROS. The present invention takes advantage of this phenomenon in a two-pronged synergistic therapeutic strategy: by enhancing the ROS level in tumor cells and by enhancing the cytotoxicity of the ROS using inducers or enhancers of ROS or compounds that activate or are activated by ROS. Examples of compounds according to the present invention that are induced or activated by ROS are certain DNA cross-linking agents disclosed herein.

According to Hergenrother, (U.S. Pat. No. 9,233,960), quinone-containing molecules are frequently cytotoxic and harm cells through two mechanisms. Many quinones are conjugate addition acceptors and readily alkylate nucleophilic species such as DNA and cysteine residues. Quinones are also substrates for 1-electron reductases, such as cytochrome P450s, cytochrome b5, xanthine oxidase, and glutathione reductase. Reduction of quinones by these enzymes generates a highly reactive semiquinone that can damage biomolecules directly, or can be oxidized by dissolved oxygen resulting in the formation of an equivalent of superoxide anion radical and the parent quinone. Thus, 1-electron reduction of quinones can catalytically create reactive oxygen species (ROS) that damage the cell.

DNQ is a potent chemotherapeutic agent exhibiting a wide therapeutic window that holds great promise for targeted therapy against a wide spectrum of difficult to treat cancers, including pancreatic and non-small cell lung cancer. See U.S. Pat. No. 9,233,960 titled “Compounds and Anti-Tumor NQO1 Substrates” incorporated in its entirety herein by reference.

Other useful Producers of NOS according to the instant invention include naphtho[2,1-d]oxazole-4,5-diones and NPDO Naphtho[1′,2′:4,5]imidazo[1,2-a]pyridine-5,6-diones which are classes of beta-lapachone analogues that are NQO1 substrates with improved aqueous solubility. Bioorg Med Chem. 2016 Mar. 1; 24(5):1006-13. doi: 10.1016/j.bmc.2016.01.024. Epub 2016 Jan. 16; Bioorg Med Chem Lett. 2016 Jan. 15; 26(2):512-7. doi: 10.1016/j.bmcl.2015.11.084. Epub 2015 Nov. 24. All of the forgoing references are incorporated in their entirety by references.

Other useful Producers of ROS include mitomycin C, EO9, RH1 (BMB Rep. 2015; 48(11): 609-617); isothiazolonaphthoquinone aulosirazole, isolated from blue-green alga (Angew Chem Int Ed Engl. 2015 Jul. 20; 54(30):8740-5. doi: 10.1002/anie.201503323. Epub 2015 Jun. 10); (±)-dunnione and its ortho-quinone (Bioorg Med Chem Lett. 2015 Mar. 15; 25(6):1244-8. doi: 10.1016/j.bmcl.2015.01.057. Epub 2015 Jan. 31); Benzofuroxans (Int J Mol Sci. 2014 Dec. 15; 15(12):23307-31. doi: 10.3390/ijms151223307); Pseudomonas aeruginosa MdaB and WrbA (J Microbiol. 2014 September; 52(9):771-7. doi: 10.1007/s12275-014-4208-8. Epub 2014 Aug. 2); 2-Substituted 3-methylnaphtho[1,2-b]furan-4,5-diones; tanshinone IIA (PLoS One. 2013 Nov. 14; 8(11):e79172. doi: 10.1371/journal.pone.0079172. eCollection 2013), Benzofuran-quinones, benzothiophene-quinones; indazole-quinones; benzisoxazole-quinones (Bioorg Med Chem. 2013 Jun. 1; 21(11): 2999-3009); 7-acetamido-2-(8′-quinolinyl)quinoline-5,8-dione, 7-amino-2-(2-pyridinyl)quinoline-5,8-dione (J Med Chem. 2013 May 23; 56(10):3806-19. doi: 10.1021/jm301689x. Epub 2013 May 1); imidazo[5,4-f]benzimidazolequinones (Bioorg Med Chem. 2012 May 15; 20(10):3223-32. doi: 10.1016/j.bmc.2012.03.063. Epub 2012 Apr. 3); lavendamycin analogues (Bioorg Med Chem. 2010 Mar. 1; 18(5):1899-909. doi: 10.1016/j.bmc.2010.01.037. Epub 2010 Jan. 25.); lavendamycin (J Med Chem. 2008 Jun. 12; 51(11):3104-15. doi: 10.1021/jm701066a. Epub 2008 May 6.); benzothiozole-quinones, and benzimidazole-quinones (Org Biomol Chem. 2007 Nov. 21; 5(22):3665-73. Epub 2007 Oct. 8). All of the forgoing references are incorporated in their entirety by references.

In addition, there are other agents that increase the amount of ROS in tumors that are useful in the invention including Longikaurin E, Chicoric acid, Celastrol, spiclomazine, TBMMP, Gemcitabine, Eriocalyxin B, Artemisinin, Genipin, P-V; MDC-1112, SKLB316, Withaferin A+oxaliplatin, Cerium oxide nanoparticles, Oleanolic acid, CDDO-Me, Belinostat, Isoalantolactone, Gallic acid, Dihydroartemisinin, BML-275, Nickel nanowires, Fenretinide, Sulforaphane, Brucein D, Artesunate, Nitric oxide-donating aspirin, Benzyl isothiocyanate, Arsenic trioxide and parthenolide, Triphala, Capsaicin, Resveratrol, and Wortmannin.

In addition, quercitin has been shown to increase ROS in leukemia and hepatoma cell lines, curcumin in neuroblastoma, vitamin C in B16 murine cells, and retinoic acid in HL60 cells all leading to apoptosis. Chul-Ho Jeong and Sang Hoon Joo: Downregulation of Reactive Oxygen Species. Journal of Cancer Prevention Vol. 21, No. 1, 2016.

As discussed above, an additional type anti-cancer agents are those that are enhanced by ROS, or pro-drugs which get activated or induced by ROS in the cancer microenvironment. The selectivity comes from the fact that many cancer cells exhibit a disturbed intracellular redox balance, making them distinctively different from their “healthy” counterparts. Among these differences, some cancer cells are hypoxic and have an increase in bioreductive processes, while others have high intracellular concentrations of reactive oxygen species (ROS) due to oxidative stress. For example, there are pro-drugs that are activated by ROS to become active DNA cross-linking agents that selectively kill cancer cells. These differences are enhanced and exploited by this invention using novel multi-faceted approaches, including therapeutically increasing the amount of ROS in cancer cells and simultaneously providing a second compound or compounds that enhance, induce or activate ROS or compounds that are enhanced, induced or activated by ROS. Such second compounds may be referred to herein without intending any limitation as “ROS Modifiers”.

Although some combinations of cancer agents have shown some improved disease control, the combinations usually have additive effects. New and more effective combinational approaches must be intelligently designed to mechanistically leverage localized drug effects to create true synergy, and doing so can lead to dramatic improvements in anti-cancer properties for such combinations and greatly improved clinical outcomes beyond what conventional chemotherapy combinations can achieve. In this present invention, we describe a novel combinational approach that is designed to create mechanistically synergistic effects for the treatment of cancer. This new design comprises of an agent that produces ROS in the disease microenvironment, in combination with at least one other agent that is enhanced, activated or induced by ROS.

Thus quinone-containing molecules alone have been shown to be low toxicity, broad spectrum anticancer agents. However, compositions of the present invention also comprise additional, often synergistic molecules that modify the reactive oxygen species created or enhanced through the addition of the NQO1 substrate molecules. These molecules include but are not limited to ROS-inducible DNA cross-linking agents. Thus, the ROS are increased preferentially in cancer cells and the apoptotic effects of these ROS molecules is magnified through the use of ROS-inducible DNA cross-linking agents.

Non-limiting examples of ROS-inducible DNA cross-linking agents have described in U.S. Pat. Nos. 8,637,490 and 8,962,670 both of which are incorporated herein in their entireties by reference and include aromatic nitrogen mustards that selectively kill cancer cells including chronic lymphocytic leukemia.

Additional examples of ROS modifying agents that can be used in conjunction with NQO1 substrates or other Producers of ROS include β-phenethyl isothiocyanate and 2-methoxyoestradiol which have been shown to selectively kill human leukemia cells but not normal lymphocytes by causing further ROS stress in cancer cells. Trachootham, D.; Zhou, Y.; Zhang, H.; Demizu, Y.; Chen, Z.; Pelicano, H.; Chiao, P. J.; Achanta, G.; Arlinghaus, R. B.; Liu, J.; Huang, P. Selective killing of oncogenically transformed cells through a ROS-mediated mechanism by beta-phenylethyl isothiocyanate. Cancer Cell 2006, 10, 241-252; and Peng, H.; Li, F.; Elizabeth, A. O.; Michael, J. K.; William, P. Superoxide dismutase as a target for the selective killing of cancer cells. Nature 2000, 407, 390-395.

An additional example of a ROS modifying agents that can be used in conjunction with NQO1 substrates is piperlongumine and its analogues which have also found to selectively kill cancer cells by increasing ROS levels but had little effect on primary normal cells. Raj, L.; Ide, T.; Gurkar, A. U.; Foley, M.; Schenone, M.; Li, X.; Tolliday, N. J.; Golub, T. R.; Carr, S. A.; Shamji, A. F.; Stern, A. M.; Mandinova, A.; Schreiber, S. L.; Lee, S. W. Selective killing of cancer cells by a small molecule targeting the stress response to ROS. Nature 2011, 475, 231-234; and Adamsa, D.; Daia, M.; Pellegrinoa, G.; Wagnera, B. K.; Sterna, A. M.; Shamjia, A. F.; Schreibera, S. L. Synthesis, cellular evaluation, and mechanism of action of piperlongumine analogs. Proc. Natl. Acad. Sci. U.S.A. 2012, 109, 15115-15120.

By the term “a compound of the quinone class” is meant any tumor cell growth inhibiting compound which is structurally related to quinone. Compounds of the quinone class include, but are not limited to, DNQ and its derivatives as well β-lapachone and its analogues. Such compounds also include, but are not limited to, any tumor cell growth inhibiting quinone analog.

Ros-activated pro-drugs include hydroxyferrocifen (J Med Chem. 2012 Jan. 26; 55(2):924-34. doi: 10.1021/jm2014937. Epub 2012 Jan. 110; Leinamycin E1 (Proc Natl Acad Sci USA. 2015 Jul. 7; 112(27):8278-83. doi: 10.1073/pnas.1506761112. Epub 2015 Jun. 8); QCA [4-(1,3,2-dioxaborinan-2-yl)benzyl ((5-methyl-2-styryl-1,3-dioxan-5-yl)methyl) carbonate] (Nat Commun. 2015 Apr. 20; 6:6907. doi: 10.1038/ncomms7907); a dual pH-sensitive PBCAE copolymer, polymeric prodrug of BCA (benzoyloxycinnamaldehyde), heme oxygenase-1 (HO-1) inhibiting zinc protoporphyrin (ZnPP)micelles (Journal of Controlled Release Volume 196, 28 Dec. 2014, Pages 19-27); aminoferrocene-based prodrugs such as N-benzylaminoferrocene (Bioorganic & Medicinal Chemistry Letters Volume 25, Issue 17, 1 Sep. 2015, Pages 3447-3450); Thiazolidinone-Based Prodrugs such as (Chem Commun (Camb). 2015 Apr. 28; 51(33): 7116-7119 doi: 10.1039/c4cc09921d); and INDQ/NO, a bioreductively activated nitric oxide prodrug (Org. Lett., 2013, 15 (11), pp 2636-2639

DOI: 10.1021/ol400884v). All of the forgoing references are incorporated in their entirety by references.

A review by Nogueira and Hay Clin Cancer Res. 2014 Aug. 15 reveals additional therapeutics which can be included in the compositions of the present invention and is therefore incorporated herein by reference in its entirety.

VELCADE (bortezomib) increases ROS. It inhibits FOXM1, which inhibits ROS, so overall effect will be an indirect increase ROS. Park H J, Carr J R, Wang Z, Nogueira V, Hay N, Tyner A L, et al. FoxM1, a critical regulator of oxidative stress during oncogenesis. Embo J. 2009; 28:2908-2918.

FOXM1 transcriptional activity and expression can also be inhibited by proteasome inhibitors such as bortezomib (velcade) and MG132, or the thiazole antibiotics, Siomycin and thiostrepton. It was shown that the suppression of FOXM1 by proteasome inhibitors sensitizes human cancer cells to cell death induced by DNA-damaging agents including doxorubicin and γ-irradiation. Bhat U G, Halasi M, Gartel A L. Thiazole antibiotics target FoxM1 and induce apoptosis in human cancer cells. PLoS One. 2009; 4:e5592. Bhat U G, Halasi M, Gartel A L. FoxM1 is a general target for proteasome inhibitors. PLoS One. 2009; 4:e6593. Radhakrishnan S K, Bhat U G, Hughes D E, Wang I C, Costa R H, Gartel A L. Identification of a chemical inhibitor of the oncogenic transcription factor forkhead box M1. Cancer Res. 2006; 66:9731-9735. Halasi M, Gartel A L. Suppression of FOXM1 sensitizes human cancer cells to cell death induced by DNA-damage. PLoS One. 2012; 7:e31761.

Disclosed in the instant invention is a whole new cancer paradigm and therapies based on escalating further the high ROS level in cancer cells to a toxic level by triggering ROS accumulation directly and/or inhibition of ROS scavenging systems represent powerful avenues for selectively killing cancer cells. In addition to those previously discussed, additional drugs have been identified as promoting ROS generation. These include: (i) mitochondrial electron transport chain modulators (e.g., arsenic trioxide, doxorubicin, topotecan); (ii) redox-cycling compounds (e.g., motexafin gadolinium); (iii) agents that disrupt the antioxidant defenses mechanism, such as GSH depleting agents (e.g., buthionine sulphoximine, D-phenylethyl isothiocyanates (PEITC)) and inhibitors of SOD (e.g., 2-methoxyestradiol), and catalase (e.g., 3-amino-1,2,4-triazole).
Trachootham D, Zhou Y, Zhang H, Demizu Y, Chen Z, Pelicano H, et al. Selective killing of oncogenically transformed cells through a ROS-mediated mechanism by beta-phenylethyl isothiocyanate. Cancer Cell. 2006; 10:241-252. [PubMed: 16959615]
Trachootham D, Alexandre J, Huang P. Targeting cancer cells by ROS-mediated mechanisms: a radical therapeutic approach? Nat Rev Drug Discov. 2009; 8:579-591. [PubMed: 19478820]
Barbieri D, Grassilli E, Monti D, Salvioli S, Franceschini M G, Franchini A, et al. D-ribose and deoxy-D-ribose induce apoptosis in human quiescent peripheral blood mononuclear cells. Biochem Biophys Res Commun. 1994; 201:1109-1116. [PubMed: 8024552]
Ceruti S, Barbieri D, Veronese E, Cattabeni F, Cossarizza A, Giammarioli A M, et al. Different pathways of apoptosis revealed by 2-chloro-adenosine and deoxy-D-ribose in mammalian astroglial cells. J Neurosci Res. 1997; 47:372-383. [PubMed: 9057130]
Xiao D, Lew K L, Zeng Y, Xiao H, Marynowski S W, Dhir R, et al. Phenethyl isothiocyanate-induced apoptosis in PC-3 human prostate cancer cells is mediated by reactive oxygen species-dependent disruption of the mitochondrial membrane potential. Carcinogenesis. 2006; 27:2223-2234. [PubMed: 16774948]

Rapamycin is a therapeutic that is useful according to the present invention to increase or induce ROS. The PI3K/AKT signaling pathway is thought to play a prominent role in the initiation and maintenance of human cancer, as many components of this pathway have been found to be mutated or amplified in a broad range of human cancers and thereby promoting resistance to therapeutic agents that induce apoptosis.

By virtue of its role in energy metabolism Akt can regulate the mitochondrial production of byproducts of energy metabolism, ROS. Akt can also regulate ROS, via its negative effects on FoxO transcription factor leading to downregulation of SOD2, catalase, and Sestrin3. Thus, the high levels of ROS as a consequence of Akt activation is due to an enhancement of mitochondrial activity as well as the downregulation of antioxidant defense mechanisms. Akt sensitizes cells to oxidative-stress induced apoptosis, by lowering the threshold of oxidative stress needed to induce cell death, and this could be exploited to selectively eradicate and to overcome chemoresistance of cancer cells with hyperactivated Akt.

Rapamycin analogs are currently being used in clinical trials and have been already approved for certain types of cancer. Rapamycin alone attenuates cell proliferation and rarely elicits cell death. Furthermore, it could also increase cell survival and chemoresistance via the inhibition of mTORC1, and consequently activating Akt through the inhibition of a negative feedback loop. However, by activating Akt, rapamycin further sensitizes cells to ROS-induced cell death, and thus, the combination of rapamycin and oxidative stress are useful according to the present invention to selectively eradicate cancer cells.

Isothiocyanates such as the PEITC are thiol modifiers that have been shown to inhibit the GSH antioxidant system by extruding GSH from the cell and by inhibiting glutathione peroxidase leading to ROS overproduction and apoptosis preferentially in cancer cells, presumably due to their increased constitutive ROS levels. Clinical studies with PEITC are currently ongoing. A combination therapy of rapamycin and PEITC was proven efficient to selectively eradicate tumors with hyperactivated Akt in pre-clinical studies. This strategy evades the chemoresistance induced by the hyperactivation of Akt in cancer cells.

Agents that enhance proteotoxic stress, including the HSP90 inhibitor IPI-504, are also known ROS inducers. It has been shown that IPI-504 and rapamycin synergize in Ras-driven tumors by promoting irresolvable ER stress, resulting in catastrophic ER and mitochondrial damage, and tumor regression. The mechanism by which these agents cooperate reveals a therapeutic paradigm that can be expanded to develop additional combinations.

It is a further object of the present invention to provide a group of pro-drugs which are of lesser cytotoxicity than the drug itself, preferably being substantially non-cytotoxic, the pro-drugs being converted in vivo under the anaerobic conditions within neoplastic tissue to the cytotoxic drug thereby mitigating the side effects of administering that drug directly. One skilled in the art would understand that efficacy and toxicity evaluations require therapeutic tradeoffs and the present invention is intended to improve that balance.

U.S. Pat. No. 8,637,490 described a group of aromatic nitrogen mustard agents that showed powerful DNA cross-linking abilities when coupled with H2O2, one of the most common ROS in cancer cells. Little DNA cross-linking was detected without H2O2. Consistent with chemistry observation, in vitro cytotoxicity assay demonstrated that these agents induced 40-80% apoptosis in primary leukemic lymphocytes isolated from CLL patients but less than 25% cell death to normal lymphocytes from healthy donors. These data provide utility and selectivity of these agents that inspires further and effective applications. Reactive Oxygen Species (ROS) Inducible DNA Cross-Linking Agents and Their Effect on Cancer Cells and Normal Lymphocytes Wenbing Chen, † t Kumudha Balakrishnan, ‡ Yunyan Kuang, † Yanyan Han, † Min Fu, ‡ Varsha Gandhi, ‡ and Xiaohua Peng*, † dx.doi.org/10.1021/jm401349g | J. Med. Chem. 2014, 57, 4498-4510.

It will be recognized by one of skill in the art that the content of the active ingredients in the pharmaceutical composition of this invention may vary quite widely depending upon numerous factors, such as, the desired dosage and the pharmaceutically acceptable carrier being employed. Physiological pH of injectables or infusion drug combinations is established by inclusion of buffering agents as is known in the pharmaceutical formulation art.

The pharmaceutically acceptable excipients described herein, for example, vehicles, adjuvants, carriers or diluents, are well-known to those who are skilled in the art and are readily available to the public. It is preferred that the pharmaceutically acceptable carrier be one that is chemically inert to the active compounds and one that has no detrimental side effects or toxicity under the conditions of use. Such pharmaceutically acceptable excipients preferably include saline (e.g., 0.9% saline), Cremophor EL (which is a derivative of castor oil and ethylene oxide available from Sigma Chemical Co., St. Louis, Mo.) (e.g., 5% Cremophor EL/5% ethanol/90% saline, 10% Cremophor EL/90% saline, or 50% Cremophor EL/50% ethanol), propylene glycol (e.g., 40% propylene glycol/10% ethanol/50% water), polyethylene glycol (e.g., 40% PEG 400/60% saline), and alcohol (e.g., 40% t-butanol/60% water). One pharmaceutical excipient for use in conjunction with the present invention is polyethylene glycol, such as PEG 400, and particularly a composition comprising 40% PEG 400 and 60% water or saline.

### Preferred Embodiments

Provided herein are methods and compositions comprising at least one first compound that increases the amount of reactive oxygen species in a disease microenvironment and at least one second compound that is activated, enhanced, or induced by reactive oxygen species. In various aspects, the methods and compositions comprises a NQO1 substrate. Preferably, the NQO1 substrate is a quinone analog. In certain aspects, the NQO1 substrate comprises DNQ or a DNQ analogue. In other aspects, the NQO1 substrate comprises beta lapachone or an analogue thereof.

Provided herein are also methods and compositions wherein the first compound comprises a compound selected from the group consisting of naphtho[2,1-d]oxazole-4,5-diones, NPDO Naphtho[1′,2′:4,5]imidazo[1,2-a]pyridine-5,6-diones, beta-lapachone, beta-lapachone analogues, mitomycin C, E09, RH1, isothiazolonaphthoquinone aulosirazole, (+)-dunnione, and the ortho-quinone of (±)-dunnione, Benzofuroxans, Pseudomonas aeruginosa MdaB and WrbA, 2-Substituted 3-methylnaphtho[1,2-b]furan-4,5-diones, tanshinone IIA, Benzofuran-quinones, benzothiophene-quinones; indazole-quinones; benzisoxazole-quinones, 7-acetamido-2-(8′-quinolinyl)quinoline-5,8-dione, 7-amino-2-(2-pyridinyl)quinoline-5,8-dione, imidazo[5,4-f]benzimidazolequinones, lavendamycin analogues, lavendamycin, benzothiozole-quinones, benzimidazole-quinones, Longikaurin E, Chicoric acid, Celastrol, spiclomazine, TBMMP, Gemcitabine, Eriocalyxin B, Artemisinin, Genipin, P-V; MDC-1112, SKLB316, Withaferin A+oxaliplatin, Cerium oxide nanoparticles, Oleanolic acid, CDDO-Me, Belinostat, Isoalantolactone, Gallic acid, Dihydroartemisinin, BML-275, Nickel nanowires, Fenretinide, Sulforaphane, Brucein D, Artesunate, Nitric oxide-donating aspirin, Benzyl isothiocyanate, Arsenic trioxide and parthenolide, Triphala, Capsaicin, Resveratrol, and Wortmannin.

Provided herein are also methods and compositions wherein the at least one second compound is selected from a drug or a pro-drug. Preferably, the pro-drug comprises a compound selected from the group consisting of hydroxyferrocifen, Leinamycin E1, [4-(1,3,2-dioxaborinan-2-yl)benzyl ((5-methyl-2-styryl-1,3-dioxan-5-yl)methyl) carbonate], a dual pH-sensitive PBCAE copolymer, a polymeric prodrug of benzoyloxycinnamaldehyde, heme oxygenase-1 inhibiting zinc protoporphyrin micelles, aminoferrocene-based prodrugs, N-benzylaminoferrocene, Thiazolidinone-Based Prodrugs, and INDQ/NO. In other aspects, the at least one second compound is selected from β-phenethyl isothiocyanate, 2-methoxyoestradiol, and piperlongumine.

In various aspects, provided herein is a composition wherein the NQO1 substrate is a DNQ analogue of the formula:

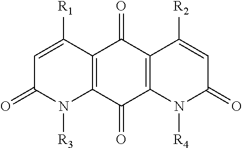

wherein
R₁ is alkyl;
R₃ is H;
R₂ and R₄ are each independently —X—R; each X is independently a direct bond or a bridging group, wherein the bridging group is —O—, —S—, —NH—, —C(—O)—, —O—C(—O)—, —C(—O)—O—, —O—C(—O)—O—, or a linker of the formula —W-A-W—, wherein
each W is independently —N(R′)C(—O)—, —C(—O)N(R)—, —OC(—O)—, —C(—O)O—, —O—, —S—, —S(O)—, —S(O)₂—, —N(R′)—, —C(—O)—, —(CH₂)ₙ— where n is 1-10, or a direct bond, wherein each R′ is independently H, (C₁-C₆)alkyl, or a nitrogen protecting group; and
each A is independently (C₁-C₂₀)alkyl, (C₂-C₁₅)alkenyl, (C₂-C₁₆)alkynyl, (C₃-C₈)cycloalkyl, (C₆-C₁₀)aryl, —(OCH₂—CH₂)ₙ— where n is 1 to about 20, —C(O)NH(CH₂)ₙ— wherein n is 1 to about 6, —OP(O)(OH)O—, —OP(O)(OH)O(CH₂)ₙ— wherein n is 1 to about 6, or (C₁-C₂₀)alkyl, (C₂-C₁₆)alkenyl, (C₂-C₁₆)alkynyl, or —(OCH₂—CH₂)ₙ— interrupted between two carbons, or between a carbon and an oxygen, with a cycloalkyl, heterocycle, or aryl group;
each R is independently alkyl, alkenyl, alkynyl, heteroalkyl, cycloalkyl, cycloalkenyl, heterocycloalkyl, heterocycloalkenyl, (cycloalkyl)alkyl, (heterocycloalkyl)alkyl, (cycloalkyl)heteroalkyl, (heterocycloalkyl)heteroalkyl, aryl, heteroaryl, (aryl)alkyl, (heteroaryl)alkyl, hydrogen, hydroxy, hydroxyalkyl, alkoxy, (alkoxy)alkyl, alkenyloxy, alkynyloxy, (cycloalkyl)alkoxy, heterocycloalkyloxy, amino, alkylamino, aminoalkyl, acylamino, arylamino, sulfonylamino, sulfinylamino, —COR^{x}, —COOR^{x}, —CONHR^{x}, —NHCOR^{x}, —NHCOOR^{x}, —NHCONHR^{x}, —N₃, —CN, —NC, —NCO, —NO₂, —SH, -halo, alkoxycarbonyl, alkylaminocarbonyl, sulfonate, sulfonic acid, alkylsulfonyl, alkylsulfinyl, arylsulfonyl, arylsulfinyl, aminosulfonyl, R^{x}S(O)R^{y}—, R^{x}S(O)₂R^{y}—, R^{x}C(O)N(R^{x})R^{y}—, R^{x}SO₂N(R^{x})R^{y}—, R^{x}N(R^{x})C(O)R—, R^{x}N(R^{x})SO₂R^{y}—, R^{x}N(R^{x})C(O)N(R^{x})R^{y}—, carboxaldehyde, acyl, acyloxy, —OPO₃H₂, —OPO₃Z₂ where Z is an inorganic cation, or saccharide; where each R^{x} is independently H, OH, alkyl or aryl, and each R^{y} is independently a group W;
wherein any alkyl or aryl can be optionally substituted with one or more hydroxy, amino, cyano, nitro, or halo groups;
or a salt or solvate thereof. In some aspects, the above composition wherein R₄ is a (C₁₋₂₀)alkyl group. In other aspects, the above composition wherein R₁ is a branched (C₁₋₂₀)alkyl group. In yet other aspects, the above composition wherein R₂ is a (C₁₋₂₀)alkyl group. In further aspects, the above composition wherein R₁ is a straight chain (C₁₋₂₀)alkyl group. In other aspects, the above composition wherein R₄ is a (C₁₋₂₀)alkyl group. In still further aspects, the above composition wherein R₁ is methyl. In yet other aspects, the above composition wherein R₂ is methyl. In further aspects, the above composition wherein R₁ and R₂ are both methyl. In other aspects, the above composition wherein R₄ is methyl.

Provided herein are also methods and compositions comprising a ROS-inducible DNA cross-linking agent plus a compound having the formula:

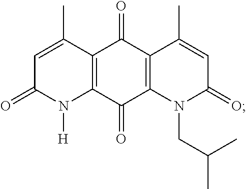 [SMILES: Cc1cc(=O)[nH]c2c1C(=O)c1c(C)cc(=O)n(CC(C)C)c1C2=O]

Additionally disclosed are methods and compositions comprising a compound having the formula:

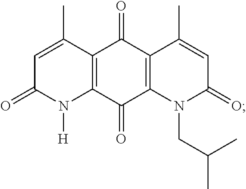 [SMILES: Cc1cc(=O)[nH]c2c1C(=O)c1c(C)cc(=O)n(CC(C)C)c1C2=O]

plus a compound having the formula

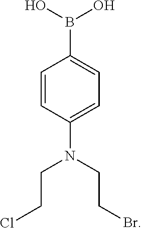 [SMILES: OB(O)c1ccc(N(CCCl)CCBr)cc1]

In further aspects, disclosed are methods and compositions comprising a compound having the formula:

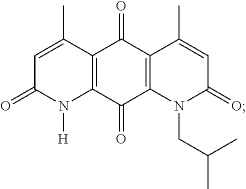 [SMILES: Cc1cc(=O)[nH]c2c1C(=O)c1c(C)cc(=O)n(CC(C)C)c1C2=O]

plus a compound having the formula:

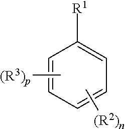

wherein:
each R′ is independently —B(XR′)₂, wherein each X is independently selected from O and S, and each R′ is independently selected from hydrogen and alkyl, or two R′ are taken together to form an optionally substituted 5- to 8-membered ring;
each R² is independently selected from optionally substituted alkyl, alkoxy, amino, halo, and —CH₂—N(R^{a})₃^{⊕};
each R³ is independently selected from:

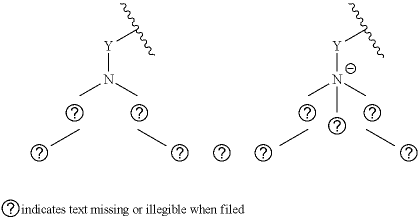

each R^{4a} and R^{4b} is independently selected from halo and —OSO₂R^{a};
each Y is independently a bond or —CH₂—;
each R⁵ is independently C₁-C₄ alkyl;
n is 0, 1 or 2;
p is 1 or 2;
each R^{a} is independently selected from optionally substituted alkyl;
wherein if the compound of formula (I) bears a positive charge, it further comprises at least one counterion Z^{⊕}.

Still additional aspects provide methods and compositions comprising a compound having the formula:

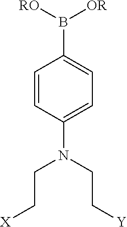

wherein
X and Y are independently selected from CL and Br and R is independently selected from 2,3-dimethylbutane and H;
plus a compound having the formula:

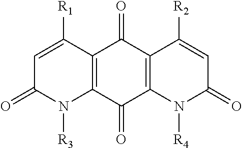

wherein
R₁ is alkyl;
R₃ is H;
R₂ and R₄ are each independently —X—R;
each X is independently a direct bond or a bridging group, wherein the bridging group is —O—, —S—, —NH—, —C(—O)—, —O—C(—O)—, —C(—O)—O—, —O—C(—O)—O—, or a linker of the formula —W-A-W—, wherein each W is independently —N(R′)C(—O)—, —C(—O)N(R)—, —OC(—O)—, —C(—O)O—, —O—, —S—, —S(O)—, —S(O)₂—, —N(R′)—, —C(—O)—, —(CH₂)ₙ— where n is 1-10, or a direct bond, wherein each R′ is independently H, (C₁-C₆)alkyl, or a nitrogen protecting group; and
each A is independently (C₁-C₂₀)alkyl, (C₂-C₁₆)alkenyl, (C₂-C₁₆)alkynyl, (C₃-C₈)cycloalkyl, (C₆-C₁₀)aryl, —(OCH₂—CH₂)ₙ— where n is 1 to about 20, —C(O)NH(CH₂)ₙ— wherein n is 1 to about 6, —OP(O)(OH)O—, —OP(O)(OH)O(CH₂)ₙ— wherein n is 1 to about 6, or (C₁-C₂₀)alkyl, (C₂-C₁₆)alkenyl, (C₂-C₁₆)alkynyl, or —(OCH₂—CH₂)ₙ— interrupted between two carbons, or between a carbon and an oxygen, with a cycloalkyl, heterocycle, or aryl group;
each R is independently alkyl, alkenyl, alkynyl, heteroalkyl, cycloalkyl, cycloalkenyl, heterocycloalkyl, heterocycloalkenyl, (cycloalkyl)alkyl, (heterocycloalkyl)alkyl, (cycloalkyl)heteroalkyl, (heterocycloalkyl)heteroalkyl, aryl, heteroaryl, (aryl)alkyl, (heteroaryl)alkyl, hydrogen, hydroxy, hydroxyalkyl, alkoxy, (alkoxy)alkyl, alkenyloxy, alkynyloxy, (cycloalkyl)alkoxy, heterocycloalkyloxy, amino, alkylamino, aminoalkyl, acylamino, arylamino, sulfonylamino, sulfinylamino, —COR^{x}, —COOR^{x}, —CONHR^{x}, —NHCOR^{x}, —NHCOOR^{x}, —NHCONHR^{x}, —N₃, —CN, —NC, —NCO, —NO₂, —SH, -halo, alkoxycarbonyl, alkylaminocarbonyl, sulfonate, sulfonic acid, alkylsulfonyl, alkylsulfinyl, arylsulfonyl, arylsulfinyl, aminosulfonyl, R^{x}S(O)R^{y}—, R^{x}S(O)₂R^{y}—, R^{x}C(O)N(R^{x})R^{y}—, R^{x}SO₂N(R^{x})R^{y}—, R^{x}N(R^{x})C(O)R^{y}—, R^{x}N(R^{x})SO₂R^{y}—, R^{x}N(R^{x})C(O)N(R^{x})R^{y}—, carboxaldehyde, acyl, acyloxy, —OPO₃H₂, —OPO₃Z₂ where Z is an inorganic cation, or saccharide; where each R^{x} is independently H, OH, alkyl or aryl, and each R^{y} is independently a group W;
wherein any alkyl or aryl can be optionally substituted with one or more hydroxy, amino, cyano, nitro, or halo groups;
or a salt or solvate thereof.

Provided herein are also methods and compositions comprising a compound having the formula:

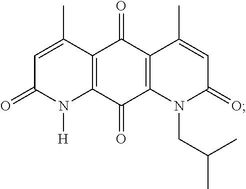 [SMILES: Cc1cc(=O)[nH]c2c1C(=O)c1c(C)cc(=O)n(CC(C)C)c1C2=O]

plus a compound having the formula:

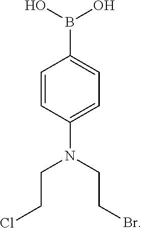 [SMILES: OB(O)c1ccc(N(CCCl)CCBr)cc1]

Provided herein are also methods and compositions comprising a compound having the formula:

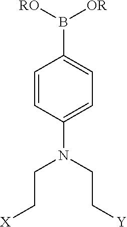

wherein
X and Y are independently selected from CL and Br and R is independently selected from 2,3-dimethylbutane and H;
plus a compound having the formula:

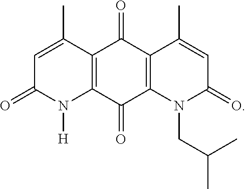 [SMILES: Cc1cc(=O)[nH]c2c1C(=O)c1c(C)cc(=O)n(CC(C)C)c1C2=O]

In various aspects provided herein are pharmaceutical compositions comprising a synergistic effective amount of a NQO1 substrate, a synergistic effective amount of an ROS inducible cytotoxin, and a pharmaceutically acceptable carrier or diluent.

Additionally, disclosed are compositions wherein the second compound is a DNA cross-linking agent. Preferably, the second compound is selected from an aromatic nitrogen mustard, β-phenethyl isothiocyanate, 2-methoxyoestradiol, and piperlongumine. Preferably, the composition further comprises an aromatic nitrogen mustard.

Other aspects of the invention provide methods of treating cancer in a subject in need of treatment, comprising administering the subject a therapeutically effective amount of the composition. Cancer types may be selected from the group consisting of leukemia, non-small cell lung cancer, colon cancer, CNS cancer, melanoma, ovarian cancer, renal cancer, prostate cancer and breast cancer.

Preferably, the methods and compositions of the invention is administered in human subjects. Preferably, the methods and compositions reduce the proliferation of a cancer cell by contacting the cancer cell with an effective amount of the composition, wherein the cancer cell is selected from the group consisting of leukemia, non-small cell lung cancer, colon cancer, CNS cancer, melanoma, ovarian cancer, renal cancer, prostate cancer and breast cancer.

More preferably, the methods and compositions of the invention provide treatment of cancer characterized by tumor cells with elevated NQO1 levels comprising administering to a patient affected by such cancer a therapeutically effective amount of one or more compositions of the invention.

What is claimed is:

1. A composition comprising at least one first compound that increases the amount of reactive oxygen species in a disease microenvironment and at least one second compound that is activated, enhanced, or induced by reactive oxygen species.

2. A composition according to claim 1 wherein the first compound comprises a NQO1 substrate.

3. A composition according to claim 2 wherein the NQO1 substrate is a quinone analog.

4. A composition according to claim 3 wherein the NQO1 substrate is DNQ or a DNQ analogue.

5. A composition according to claim 1 wherein the NQO1 substrate is beta lapachone or an analogue thereof.

6. A composition according to claim 1 wherein the first compound comprises a compound selected from the group consisting of naphtho[2,1-d]oxazole-4,5-diones, NPDO Naphtho[1′,2′:4,5]imidazo[1,2-a]pyridine-5,6-diones, beta-lapachone, beta-lapachone analogues, mitomycin C, E09, RH1, isothiazolonaphthoquinone aulosirazole, (±)-dunnione, and the ortho-quinone of (±)-dunnione, Benzofuroxans, Pseudomonas aeruginosa MdaB and WrbA, 2-Substituted 3-methylnaphtho[1,2-b]furan-4,5-diones, tanshinone IIA, Benzofuran-quinones, benzothiophene-quinones; indazole-quinones; benzisoxazole-quinones, 7-acetamido-2-(8′-quinolinyl)quinoline-5,8-dione, 7-amino-2-(2-pyridinyl)quinoline-5,8-dione, imidazo[5,4-f]benzimidazolequinones, lavendamycin analogues, lavendamycin, benzothiozole-quinones, benzimidazole-quinones, Longikaurin E, Chicoric acid, Celastrol, spiclomazine, TBMMP, Gemcitabine, Eriocalyxin B, Artemisinin, Genipin, P-V; MDC-1112, SKLB316, Withaferin A+oxaliplatin, Cerium oxide nanoparticles, Oleanolic acid, CDDO-Me, Belinostat, Isoalantolactone, Gallic acid, Dihydroartemisinin, BML-275, Nickel nanowires, Fenretinide, Sulforaphane, Brucein D, Artesunate, Nitric oxide-donating aspirin, Benzyl isothiocyanate, Arsenic trioxide and parthenolide, Triphala, Capsaicin, Resveratrol, and Wortmannin.

7. A composition according to any one of claims 1 through 6 wherein the at least one second compound is selected from a drug or a pro-drug.

8. A composition according to claim 7 wherein the pro-drug comprises a compound selected from the group consisting of hydroxyferrocifen, Leinamycin E1, [4-(1,3,2-dioxaborinan-2-yl)benzyl ((5-methyl-2-styryl-1,3-dioxan-5-yl)methyl) carbonate], a dual pH-sensitive PBCAE copolymer, a polymeric prodrug of benzoyloxycinnamaldehyde, heme oxygenase-1 inhibiting zinc protoporphyrin micelles, aminoferrocene-based prodrugs, N-benzylaminoferrocene, Thiazolidinone-Based Prodrugs, and INDQ/NO.

9. A composition according to any one of claims 1 through 6 wherein the at least one second compound is selected from β-phenethyl isothiocyanate, 2-methoxyoestradiol, and piperlongumine.

10. A composition according to claim 2 wherein the NQO1 substrate is a DNQ analogue of the formula: 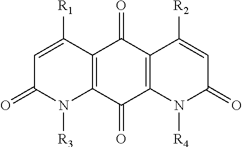
wherein
R₁ is alkyl;
R₃ is H;
R₂ and R₄ are each independently —X—R;
each X is independently a direct bond or a bridging group, wherein the bridging group is —O—, —S—, —NH—, —C(—O)—, —O—C(—O)—, —C(—O)—O—, —O—C(—O)—O—, or a linker of the formula —W-A-W—, wherein each W is independently —N(R′)C(—O)—, —C(—O)N(R)—, —OC(—O)—, —C(—O)O—, —O—, —S—, —S(O)—, —S(O)₂—, —N(R′)—, —C(—O)—, —(CH₂)ₙ— where n is 1-10, or a direct bond, wherein each R′ is independently H, (C₁-C₆)alkyl, or a nitrogen protecting group; and
each A is independently (C₁-C₂₀)alkyl, (C₂-C₁₆)alkenyl, (C₂-C₁₆)alkynyl, (C₃-C₈)cycloalkyl, (C₆-C₁₀)aryl, —(OCH₂—CH₂)ₙ— where n is 1 to about 20, —C(O)NH(CH₂)ₙ— wherein n is 1 to about 6, —OP(O)(OH)O—, —OP(O)(OH)O(CH₂)ₙ— wherein n is 1 to about 6, or (C₁-C₂₀)alkyl, (C₂-C₁₆)alkenyl, (C₂-C₁₆)alkynyl, or —(OCH₂—CH₂)ₙ— interrupted between two carbons, or between a carbon and an oxygen, with a cycloalkyl, heterocycle, or aryl group;
each R is independently alkyl, alkenyl, alkynyl, heteroalkyl, cycloalkyl, cycloalkenyl, heterocycloalkyl, heterocycloalkenyl, (cycloalkyl)alkyl, (heterocycloalkyl)alkyl, (cycloalkyl)heteroalkyl, (heterocycloalkyl)heteroalkyl, aryl, heteroaryl, (aryl)alkyl, (heteroaryl)alkyl, hydrogen, hydroxy, hydroxyalkyl, alkoxy, (alkoxy)alkyl, alkenyloxy, alkynyloxy, (cycloalkyl)alkoxy, heterocycloalkyloxy, amino, alkylamino, aminoalkyl, acylamino, arylamino, sulfonylamino, sulfinylamino, —COR^{x}, —COOR^{x}, —CONHR^{x}, —NHCOR^{x}, —NHCOOR^{x}, —NHCONHR^{x}, —N₃, —CN, —NC, —NCO, —NO₂, —SH, -halo, alkoxycarbonyl, alkylaminocarbonyl, sulfonate, sulfonic acid, alkylsulfonyl, alkylsulfinyl, arylsulfonyl, arylsulfinyl, aminosulfonyl, R^{x}S(O)R^{y}—, R^{x}S(O)₂R^{y}—, R^{x}C(O)N(R^{x})R^{y}—, R^{x}SO₂N(R^{x})R^{y}—, R^{x}N(R^{x})C(O)R^{y}—, R^{x}N(R^{x})SO₂R^{y}—, R^{x}N(R^{x})C(O)N(R^{x})R^{y}—, carboxaldehyde, acyl, acyloxy, —OPO₃H₂, —OPO₃Z₂ where Z is an inorganic cation, or saccharide; where each R^{x} is independently H, OH, alkyl or aryl, and each R^{y} is independently a group W;
wherein any alkyl or aryl can be optionally substituted with one or more hydroxy, amino, cyano, nitro, or halo groups;
or a salt or solvate thereof.

11. The composition of claim 10 wherein R₄ is a (C₁₋₂₀)alkyl group.

12. The composition of claim 10 wherein R₁ is a branched (C₁₋₂₀)alkyl group.

13. The composition of claim 10 wherein R₂ is a (C₁₋₂₀)alkyl group.

14. The composition of claim 10 wherein R₁ is a straight chain (C₁₋₂₀)alkyl group.

15. The composition of claim 10 wherein R₄ is a (C₁₋₂₀)alkyl group.

16. The composition of claim 10 wherein R₁ is methyl.

17. The composition of claim 10 wherein R₂ is methyl.

18. The composition of claim 10 wherein R₁ and R₂ are both methyl.

19. The composition of claim 10 wherein R₄ is methyl.

20. A composition comprising a ROS-inducible DNA cross-linking agent plus a compound having the formula

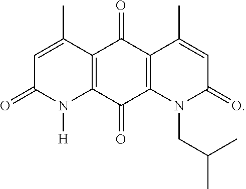 [SMILES: Cc1cc(=O)[nH]c2c1C(=O)c1c(C)cc(=O)n(CC(C)C)c1C2=O]

21. A composition comprising a compound having the formula 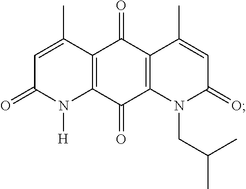 [SMILES: Cc1cc(=O)[nH]c2c1C(=O)c1c(C)cc(=O)n(CC(C)C)c1C2=O]
plus a compound having the formula

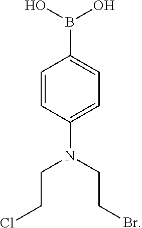 [SMILES: OB(O)c1ccc(N(CCCl)CCBr)cc1]

22. A composition comprising a compound having the formula 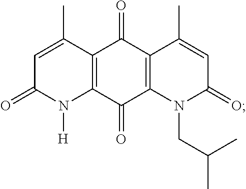 [SMILES: Cc1cc(=O)[nH]c2c1C(=O)c1c(C)cc(=O)n(CC(C)C)c1C2=O]
plus a compound having the formula 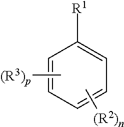
wherein:
each R¹ is independently —B(XR′)₂, wherein each X is independently selected from O and S, and each R′ is independently selected from hydrogen and alkyl, or two R′ are taken together to form an optionally substituted 5- to 8-membered ring;
each R² is independently selected from optionally substituted alkyl, alkoxy, amino, halo, and —CH₂—N(R^{a})₃^{⊕};
each R³ is independently selected from: 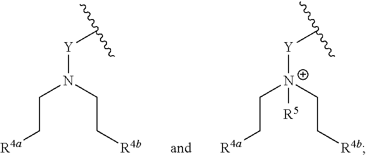
each R^{4a} and R^{4b} is independently selected from halo and —OSO₂R^{a};
each Y is independently a bond or —CH₂—;
each R⁵ is independently C₁-C₄ alkyl;
n is 0, 1 or 2;
p is 1 or 2;
each R^{a} is independently selected from optionally substituted alkyl;
wherein if the compound of formula (I) bears a positive charge, it further comprises at least one counterion Z^{⊕}.

23. A composition comprising a compound having the formula 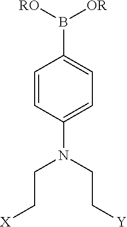
wherein
X and Y are independently selected from CL and Br and R is independently selected from 2,3-dimethylbutane and H;
plus a compound having the formula 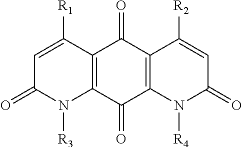
wherein
R₁ is alkyl;
R₃ is H;
R₂ and R₄ are each independently —X—R;
each X is independently a direct bond or a bridging group, wherein the bridging group is —O—, —S—, —NH—, —C(—O)—, —O—C(—O)—, —C(—O)—O—, —O—C(—O)—O—, or a linker of the formula —W-A-W—, wherein each W is independently —N(R′)C(—O)—, —C(—O)N(R)—, —OC(—O)—, —C(—O)O—, —O—, —S—, —S(O)—, —S(O)₂—, —N(R′)—, —C(—O)—, —(CH₂)ₙ— where n is 1-10, or a direct bond, wherein each R′ is independently H, (C₁-C₆)alkyl, or a nitrogen protecting group; and
each A is independently (C₁-C₂₀)alkyl, (C₂-C₁₆)alkenyl, (C₂-C₁₆)alkynyl, (C₃-C₈)cycloalkyl, (C₆-C₁₀)aryl, —(OCH₂—CH₂)ₙ— where n is 1 to about 20, —C(O)NH(CH₂)ₙ— wherein n is 1 to about 6, —OP(O)(OH)O—, —OP(O)(OH)O(CH₂)— wherein n is 1 to about 6, or (C₁-C₂₀)alkyl, (C₂-C₁₆)alkenyl, (C₂-C₁₆)alkynyl, or —(OCH₂—CH₂)ₙ— interrupted between two carbons, or between a carbon and an oxygen, with a cycloalkyl, heterocycle, or aryl group;
each R is independently alkyl, alkenyl, alkynyl, heteroalkyl, cycloalkyl, cycloalkenyl, heterocycloalkyl, heterocycloalkenyl, (cycloalkyl)alkyl, (heterocycloalkyl)alkyl, (cycloalkyl)heteroalkyl, (heterocycloalkyl)heteroalkyl, aryl, heteroaryl, (aryl)alkyl, (heteroaryl)alkyl, hydrogen, hydroxy, hydroxyalkyl, alkoxy, (alkoxy)alkyl, alkenyloxy, alkynyloxy, (cycloalkyl)alkoxy, heterocycloalkyloxy, amino, alkylamino, aminoalkyl, acylamino, arylamino, sulfonylamino, sulfinylamino, —COR^{x}, —COOR^{x}, —CONHR^{x}, —NHCOR^{x}, —NHCOOR^{x}, —NHCONHR^{x}, —N₃, —CN, —NC, —NCO, —NO₂, —SH, -halo, alkoxycarbonyl, alkylaminocarbonyl, sulfonate, sulfonic acid, alkylsulfonyl, alkylsulfinyl, arylsulfonyl, arylsulfinyl, aminosulfonyl, R^{x}S(O)R^{y}—, R^{x}S(O)₂R^{y}—, R^{x}C(O)N(R^{x})R^{y}—, R^{x}SO₂N(R^{x})R^{y}—, R^{x}N(R^{x})C(O)R^{y}—, R^{x}N(R^{x})SO₂R^{y}—, R^{x}N(R^{x})C(O)N(R^{x})R^{y}—, carboxaldehyde, acyl, acyloxy, —OPO₃H₂, —OPO₃Z₂ where Z is an inorganic cation, or saccharide; where each R^{x} is independently H, OH, alkyl or aryl, and each R^{y} is independently a group W;
wherein any alkyl or aryl can be optionally substituted with one or more hydroxy, amino, cyano, nitro, or halo groups;
or a salt or solvate thereof.

24. A composition according to claim 23 comprising a compound having the formula 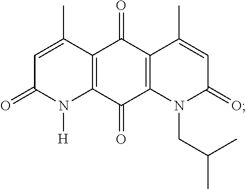 [SMILES: Cc1cc(=O)[nH]c2c1C(=O)c1c(C)cc(=O)n(CC(C)C)c1C2=O]
plus a compound having the formula

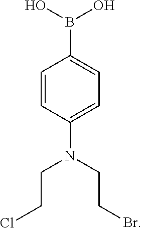 [SMILES: OB(O)c1ccc(N(CCCl)CCBr)cc1]

25. A composition comprising a compound having the formula 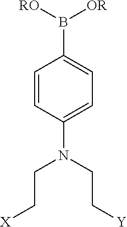
wherein
X and Y are independently selected from CL and Br and R is independently selected from 2,3-dimethylbutane and H;
plus a compound having the formula

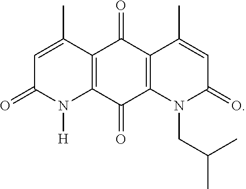 [SMILES: Cc1cc(=O)[nH]c2c1C(=O)c1c(C)cc(=O)n(CC(C)C)c1C2=O]

26. A pharmaceutical composition comprising a synergistic effective amount of a NQO1 substrate, a synergistic effective amount of an ROS inducible cytotoxin, and a pharmaceutically acceptable carrier or diluent.

27. A composition according to claim 1 wherein the second compound is a DNA cross-linking agent.

28. A composition according to claim 1 wherein the second compound is selected from an aromatic nitrogen mustard, 1-phenethyl isothiocyanate, 2-methoxyoestradiol, and piperlongumine.

29. A composition according to claim 5 further comprising an aromatic nitrogen mustard.

30. A method of treating cancer in a subject in need of treatment, comprising administering the subject a therapeutically effective amount of a composition of claim 1, wherein the cancer is selected from the group consisting of leukemia, non-small cell lung cancer, colon cancer, CNS cancer, melanoma, ovarian cancer, renal cancer, prostate cancer and breast cancer.

31. The method of claim 30 wherein the subject is a human.

32. A method of reducing the proliferation of a cancer cell, comprising contacting the cancer cell with an effective amount of a composition of claim 1, wherein the cancer cell is selected from the group consisting of leukemia, non-small cell lung cancer, colon cancer, CNS cancer, melanoma, ovarian cancer, renal cancer, prostate cancer and breast cancer.

33. A method of treating cancer characterized by tumor cells with elevated NQO1 levels comprising administering to a patient affected by such cancer a therapeutically effective amount of a composition selected from the compounds of any one of claims 1 through 31.
